(19) United States
(12) Patent Application Publication
(10) Pub. No.: US 2015/0268234 A1
(43) Pub. Date: Sep. 24, 2015

(54) Density Amplification in Magnetic Levitation to Detect Binding Events of Large Molecules

(71) Applicant: President and Fellows of Harvard College, Cambridge, MA (US)

(72) Inventors: George M. Whitesides, Newton, MA (US); Nathan D. Shapiro, Cambridge, MA (US); Anand Bala Subramaniam, Cambridge, MA (US)

(21) Appl. No.: 14/417,252

(22) PCT Filed: Jul. 29, 2013
(86) PCT No.: PCT/US13/52485

§ 371 Date: Jan. 26, 2015

Related U.S. Application Data
(60) Provisional application No. 61/676,649, filed on Jul. 27, 2012.

(51) Int. Cl. G01N 33/543; G01N 33/571; G01N 33/569; G01N 33/553

(52) U.S. Cl. CPC G01N 33/54306; G01N 33/54373; G01N 33/553; G01N 33/571; G01N 33/56988

(57) ABSTRACT

A system and method that quantifies the concentration of an immunoactive analyte by detecting a chemically amplified change in density. This method, termed DeLISA for Density-Linked Immunosorbent Assay, but useful for any biomolecular recognition event, uses magnetic levitation (MagLev) to detect the changes in density. The present disclosure provides a quantitative measure of detecting binding events, does not require the use of electricity, and can be easily multiplexed to detect multiple analytes since several beads can be placed in single serum sample to detect, for example, HIV, Syphilis, Hepatitis C, and the like, simultaneously.

### CROSS-REFERENCES TO RELATED APPLICATIONS

This application claims the benefit of the earlier filing date of U.S. Patent Application No. 61/676,649, filed on Jul. 27, 2012, the contents of which is incorporated by reference herein in its entirety.

### BACKGROUND

Immunoassays are widely employed biochemical tests capable of detecting the presence of an analyte in a liquid sample with high sensitivity and specificity. At its core, this technique measures the binding of antibodies to specific antigens. Signal amplification is important in immunoassays to translate molecular binding events into an accessible readout. Typical amplification and readout schemes employed in immunoassays are based on colorimetry, fluorimetry, optical densitometry, chemiluminescence or electrochemistry.

### SUMMARY

Colorimetric amplification, by far the most common and simplest to implement, is subjective and often not quantitative when performed in non-laboratory settings. Other extant methods, while potentially more sensitive and quantitative, typically require specialized equipment. In addition, most of these methods are difficult to multiplex.

A system and method that quantifies the concentration of an analyte by detecting a chemically amplified change in density is described. This method, termed DeLISA for Density-Linked Immununosorbent Assay (but not specifically limited to immunoassays), uses magnetic levitation (MagLev) to detect the changes in density. DeLISA provides a quantitative measure of detecting binding events, does not require the use of electricity, and can be easily multiplexed to detect multiple analytes since several beads can be placed in single serum sample to detect, for example, HIV, syphilis Hepatitis C, and the like, simultaneously.

### BRIEF DESCRIPTION OF DRAWINGS

FIG. 1A shows a magnetic levitation device, according to an embodiment of the present disclosure.

FIG. 1B schematically depicts a magnetic levitation device, according to an embodiment of the present disclosure.

FIG. 1C provides a flow chart for amplifying the density change to allow detection of antibody binding according to certain embodiments of the present disclosure.

FIG. 1D shows density changes that occur over longer periods of chemical amplification according to an embodiment of the present disclosure.

FIG. 2 shows a schematic of an assay procedure for mass density-linked immunosorbent assay (DeLISA), according to an embodiment of the present disclosure.

FIG. 3 shows a schematic of a DeLISA experimental procedure, according to an embodiment of the present disclosure.

FIG. 4A shows photographs of beads in a MagLev device for detecting varying concentrations of goat anti-HIV-1 p24, according to an embodiment of the present disclosure. PS beads were coated with p24 and incubated in buffered liquid samples containing varying concentrations of goat anti-HIV-1 p24. After completion of immune recognition events the beads were treated with a silver density amplification solution (Sigma-Aldrich) for 25 minutes. The shown photographs are of the heads in the MagLev device following silver amplification.

FIG. 4B shows a plot of the average change in levitation height versus concentration of anti-HIV-1 p24 (log scale) in a MagLev device, according to an embodiment of the present disclosure. The error bars indicate one standard deviation in the mean, calculated from a single experiment.

FIG. 5 shows a schematic of an assay procedure for mass density-linked immunosorbent assay (DeLISA), according to an embodiment of the present disclosure.

FIG. 6A shows photographs of beads in a MagLev device following silver amplification using DeLISA to detect anti-HIV-1 p24 from liquid samples, according to an embodiment of the present disclosure. PS beads were coated with p24 and incubated in buffered liquid samples containing varying concentrations of goat anti-HIV-1 p24. After completion of immune recognition events the beads were treated with a silver density amplification solution (Sigma-Aldrich) for 25 minutes.

FIG. 6B shows a plot of the average change in levitation height in a MagLev device versus the concentration of anti-HIV-1 p24 (log scale) using DeLISA, according to an embodiment of the present disclosure.

FIG. 7 shows photographs of a quantitative singleplex DeLISA assay for HIV1 antibodies in serum, according to an embodiment of the present disclosure. The colored beads were exposed to samples of varying concentration of HIV-1 antibodies and silver amplified for 18 minutes.

FIG. 8A shows photographs from a multiplex DeLISA assay, according to an embodiment of the present disclosure.

FIG. 8B shows a table of results from a multiplex DeLISA assay, according to an embodiment of the present disclosure.

FIG. 9A shows a photograph of spheres in a DeLISA assay with the density amplification step performed with silver, according to an embodiment of the present disclosure.

FIG. 9B shows a photograph of spheres in a DeLISA assay with the density amplification step performed with gold, according to an embodiment of the present disclosure.

FIG. 10 shows a photograph of Kapton sheets used in a DeLISA assay, according to an embodiment of the present disclosure. Scale bar is 500 mm.

FIG. 11A shows photographs of a quantitative DeLISA assay for Syphilis+ve goat serum, according to an embodiment of the present disclosure.

FIG. 11B shows photographs of a quantitative DeLISA assay for disease free goat serum, according to an embodiment of the present disclosure.

FIG. 12 shows photographs of a quantitative DeLISA assay for HIV antibodies after 4, 12, and 25 minutes, according to an embodiment of the present disclosure.

FIG. 13A shows a schematic illustration of carrying out DeLISA using a single head in a single capillary, according to an embodiment of the present disclosure.

FIG. 13B shows images of DeLISA at different amplification times according to an embodiment of the present disclosure.

FIG. 14A shows a plot of normalized levitation height as a function of time according to an embodiment of the present disclosure.

FIG. 14B shows a plot of normalized intensity as a function of time according to an embodiment of the present disclosure.

### DETAILED DESCRIPTION

In immunology, each antibody binds to a specific antigen by way of an interaction similar to the fit between a lock and a key. This interaction is part of the immune system's response to try to destroy or neutralize any antigen that is recognized as a foreign and potentially harmful invader (e.g., viruses, bacteria, etc). Hence, it is important to be able to detect the presence of certain antibodies in a sample.

Most immunoassay readouts involve optical detection. Examples include detecting, (i) a change in the intensity of impinging light due to absorbance, (ii) a change in the wavelength of impinging light (color change), or, (iii) the production of light from fluorescence or chemiluminescence. While these techniques are broadly useful, they often require sophisticated equipment and may be sensitive to observation conditions. A low-cost, easy-to-use alternative would be valuable for specific applications (for example, in point-of-care diagnosis and/or in resource-limited settings).

Enzyme-linked immunosorbent assays (ELISAs) are standard diagnostic tools. ELISA is a popular format of an analytic biochemistry assay that uses a solid-phase enzyme immunoassay (EIA) to detect the presence of a substance, usually an antigen, in a liquid sample or wet sample. Antigens from the sample are attached to the substrate. Then, a further specific antibody is applied over the surface so it can bind to the antigen. This antibody is linked to an enzyme, and, in the final step, a substance containing the enzyme's substrate is added. The subsequent reaction produces a detectable signal, most commonly a color change in the substrate. Variants of ELISA can be found in common consumer diagnostics such as pregnancy tests as well as in point-of-care tools for the diagnosis of viral infections.

In certain embodiments of the present disclosure, new method for conducting an immunoassay is described. Specifically, a new approach to the visualization of the result of an immunoassay is demonstrated. In one embodiment, this approach allows for amplification and quantification of antigen-antibody binding events based on changes in the density of a substrate.

Although there are many types of immunoassays which use different techniques to detect the presence of specific analytes, the present disclosure utilizes a chemically amplified change in density to quantify antigen-antibody binding events.

While some embodiments of this disclosure use a chemically amplified change in density to measure a change in the levitation height of a substrate to conduct immunoassays, most immunoassays monitor a change in color, fluorescence etc. which can be somewhat subjective. A change in levitation height is a more objective means of obtaining a readout. Furthermore, other issues with current amplification techniques include, complex equipment for readout and arc difficult to multiplex.

In one embodiment, the method can use a chemically amplified change in density of substrates, such as beads, colloidal particles, spheres, flat substrates, and the like, having on their surface a desired amount of density amplification material, such as gold-labeled or silver-labeled antibodies, to quantify antigen-antibody binding events (e.g., silver or gold deposits onto the beads that have antibodies hound onto its surface causing a change in density).

In one embodiment, the present disclosure uses magnetic levitation (MagLev) to detect the changes in density. This method quantifies the concentration of an analyte by detecting a change in the levitation height of an immunosorbed material in a MagLev device. This presents a new approach to the visualization of the result of an immunoassay thorough MagLev, and is referred to as Density Linked Immunosorbent Assay (DeLISA) in this disclosure.

DeLISA should be of interest to the diagnostic community due to the ease with which it can be multiplexed. Moreover, DeLISA presents a unique way of conducting and quantifying immunoassays: measuring macroscopic changes in height due to molecular level immune recognition events.

Although DeLISA is provided as one particular example, additional biomolecular recognition events can be monitored using the disclosure provided herein, such as binding of nucleic acids (e.g., DNA), antibody fragments, proteins (e.g., streptavidin-biotin), and the like.

### MagLev

In general, the principle of magnetic levitation involves subjecting materials of having different densities (or which develop different densities over time) in a fluid medium having paramagnetic or superparamagnetic properties to an inhomogeneous magnetic field, as described in, for example, in PCT Application No. US08/68797 entitled “Density-Based Methods For Separation Of Materials, Monitoring Of Solid Supported Reactions And Measuring Densities Of Small Liquid Volumes And Solids,” filed on Jun. 30, 2008, the contents of which is incorporated by reference herein in its entirety. As described therein, MagLev devices can be constructed so that particles of higher density ‘sink’ when placed in the magnetic field while particles of lower density ‘float’. This phenomenon can be used to detect particle composition, density, and other properties based on their characteristic location in a magnetic fluid.

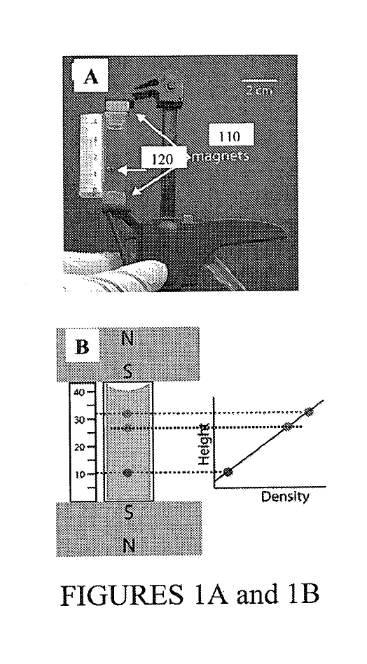

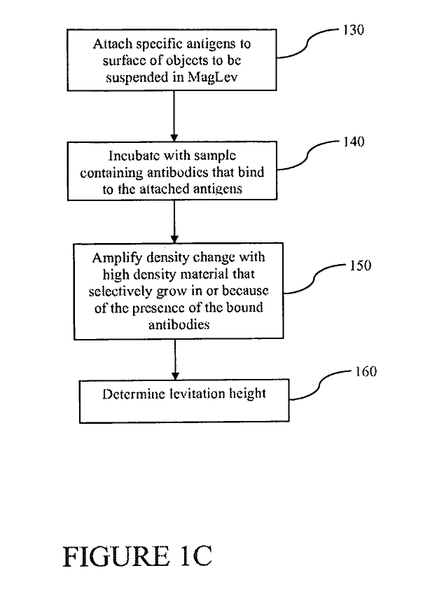

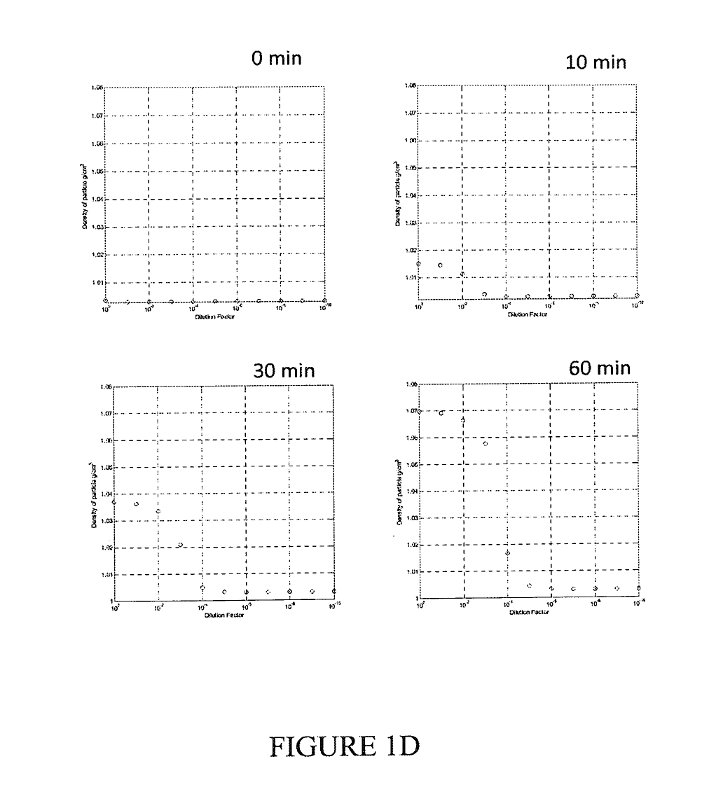

Most substrates are diamagnetic, and are repelled by magnetic fields. The effect is usually small, unless substrates are surrounded by a paramagnetic fluid (e.g., Mn2+, Gd3+ ions in solution), in which case, as shown in FIG. 1A, inexpensive portable magnets 110 can be used to levitate substrates 120. When placed in the paramagnetic fluid, the diamagnetic substrates levitate at a height that corresponds to their density (FIG. 1B).

MagLev provides a fundamentally different way of conducting an immunoassay. MagLev translates changes in the mass density of an arbitrary substrate into easily understood, one-dimensional changes in levitation height. Certain embodiments of MagLev have a number of advantageous properties, including: (i) it requires no electricity and a minimal amount of laboratory equipment; typically, a cuvette filled with a paramagnetic solution, and two relatively inexpensive NdFeB magnets ($5-20 each) oriented with like poles facing each other; (ii) it is easy to use and the results can be quantified unambiguously; (iii) it is sensitive; changes in density on the order of about ±0.0005 g/mL are easily detected under the levitation conditions employed (200-300 mM MnCl₂), even changes in density on the order of about ±0.0002 g/cm³ can be detected; (iv) it can be multiplexed by using color-coded or differently shaped solid supports so that it is operationally very easy to quantify the concentration of multiple antigens or antibodies simultaneously; and (v) exotic configurations such as tilting the magnets, can be employed to increase sensitivity. Thus the disclosed methods and approaches have the potential to be a low-cost, field accessible diagnostic tool.

Despite the advantages of MagLev in discerning differences in density among different samples, certain challenges remain. For example, despite the high sensitivity of the MagLev technique, it was unexpectedly found that binding events of large molecules, such as binding of antibodies (or antibody fragments) to specific antigens placed on the surface of substrates suspended in a paramagnetic liquid, antigens to specific antibodies placed on the surface of substrates suspended in a paramagnetic liquid, nucleic acids to complementary nucleic acids placed on the surface of substrates suspended in a paramagnetic liquid or proteins to complementary proteins placed on the surface of substrates suspended in a paramagnetic liquid, produce no measurable change in the levitation height.

Moreover, to be useful as a diagnostic tool to detect the binding of antibodies to antigens, detection of very low concentration of antibodies in solution, such as less than 200 nM are needed. This further adds to the difficulty of detecting antibody binding events as there may be an insufficient amount of antibodies in solution to cause a measurable change in density.

Previous attempts to enhance sensitivity of binding events in MagLev include binding the analyte to the surface of the suspended substrate to add mass without significantly changing the volume of the suspended substrate. However, binding of antibodies are not susceptible to such efforts as they are large molecules having specific conformations that measurably increase the particle volume as well as mass in a binding event.

Another attempt to enhance sensitivity of binding events in MagLev include using macroporous substrates, where the binding events are carried out within the pores of the substrate. When binding occurs within the pores of the macroporous substrate, the substrate mass increases, without volume change, resulting a density increase. As before, the technique involved increasing mass without changing in the overall volume of the suspended substrate. However, detecting binding of antibodies (or the like) using this technique also proved ineffective as the antibodies were too large and diffusion of the antibodies into the pores of the substrates rarely occurred or occurred too slowly.

Hence, conventional MagLev approaches to detecting binding events of large molecules, such as antibodies, nucleic acids, proteins, and the like were not possible.

### DeLISA.

The present disclosure, referred to herein as “DeLISA,” (but not limited only to immunoassay but applicable to other biomolecular binding events) provides a way to expand the sensitivity of binding events in MagLev, particularly useful as a diagnostic tool for detecting the presence of antibodies in a sample. As shown in FIG. 1C, the approach involves providing on a surface of a substrate antigens that are able to selectively bind to the antibodies of interest (130). Then, the substrate having antigens are incubated with sample containing antibodies that bind to the attached antigens (140). In 150, the density change is amplified by adding a density amplification material having a density that is significantly different from that of the suspended substrate that selectively grows in or near the presence of the bound antibodies (150). In certain embodiments, the density amplification material can be added through a multi-step process where secondary moieties are attached to the bound antibody. The secondary moieties can include secondary antibodies, seed particles, catalysts, or combinations thereof that aid in the addition of the density amplification material. Then, the levitation height of the chemically amplified substrates can be measured (160). The density amplification step 150 increases the density difference between the unbound and bound antigen:antibody conjugate to provides a measurable difference in the levitation height as compared to the levitation height of the substrates that have not been chemically amplified.

### Substrates

Many different substrates can be utilized. Generally, any diamagnetic material having a surface that can bind antigens and/or antibodies can be utilized as a substrate for the Immunosorbent Assay. In certain embodiments, the diamagnetic material can be treated to allow attachment of antigens to the surface thereof in certain embodiments, the diamagnetic material can be treated to allow attachment of antibodies to the surface thereof. The substrate is diamagnetic so that it does not influence the equilibrium position Of the particle in the magnetic field.

Some exemplary objects that can be utilized include diamagnetic particles, sheets, rods, cylinders, and the like. Some exemplary material include polymers, such as polystyrene (PS), polymethylmethacrylate (PMMA), agarose, nylon, paper, nitrocellulose, Immunodyne, and the like.

In certain embodiments, diamagnetic substrates having a size of less than 600 microns, 500 microns, 400 microns, 300 microns, or even 50 microns may be utilized. While larger substrates may be utilized, smaller substrates may allow the detection of antigen-antibody binding to be carried out within time limits typically considered acceptable for diagnostic assays. For example, the density changing amplification can proceed in a manner such that appreciable change in levitation height can be detected within 1 hour or less of amplification.

### Antigens

Many different antigens can be utilized, depending on the particular assay involved. Some exemplary antigens that can be attached to the substrate include HIV p24 antigen, Syphilis p41 antigen, Hepatitis Core antigen, Rubella virus antigen, haptens of antibiotics, haptens of penicillin, haptens of ampicillin, haptens of chloramphenicol, and others.

In certain embodiments, if detection of antibodies in a test solution is desired, antigens can be bound onto the surface of a substrate. Binding of specific antigens onto the surface of a substrate can be carried out using various different techniques, such as those described in the examples below. Other well-known techniques, that would be readily apparent to one of ordinary skill in the art, can also be utilized.

In alternative embodiments, antigens can present in a test solution to be detected so that they can be specifically bound to antibodies that have been bound onto the surface of a substrate. Such attachment to specific antibodies are known to one of skilled in the art.

In certain embodiments, the antigens can be present in a test solution in low concentrations, such as less than 200 nM, less than 100 nM, less than 50 nM, less than 10 less than 2 nM, less than 1 nM, less than 500 pM, less than 200 pM, less than 100 pM, less than 50 pM, less than 10 pM, or even as low as a few pM (e.g., 2 pM).

In certain embodiments, the antigens can be in a test solution that is miscible with the paramagnetic liquid used in MagLev. For such antigen test solutions, binding of the antigens to the antibodies bound on the surface of the substrates can be carried out within the DeLISA equipment.

In other embodiments, the antigens can be in a test solution that is not miscible with the paramagnetic liquid used in MagLev. For such antigen test solutions, binding of the antigens to the antibodies bound on the surface of the substrates can be carried out outside of the DeLISA equipment, the bound substrates recovered and introduced into the subsequent density amplification environment and/or the MagLev equipment.

### Antibodies

Many different antibodies can be utilized, depending on the particular assays involved. In certain embodiments, HIV, Syphilis, Hepatitis C, rubella, antibiotics, penicillin, ampicillin, chloramphenicol, and other desired antibodies can be utilized or detected.

In certain embodiments, if detection of antibodies in a test solution is desired, antibodies can be present in a test solution to be detected so that they can be specifically bound to antigens that have been bound onto the surface of a substrate. Such attachment to specific antigens are known to one of skilled in the art.

In certain embodiments, the antibodies can be present in a test solution in low concentrations, such as less than 200 nM, less than 100 nM, less than 50 nM, less than 10 nM, less than 2 nM, less than 1 nM, less than 500 pM, less than 200 pM, less than 100 pM, less than 50 pM, less than 10 pM, or even as low as a few pM (e.g., 2 pM).

In certain embodiments, the antibodies can be in a test solution that is miscible with the paramagnetic liquid used in MagLev. For such antibody test solutions, binding of the antibodies to the antigens bound on the surface of the substrates can be carried out within the DeLISA equipment.

In other embodiments, the antibodies can be in a test solution that is not miscible with the paramagnetic liquid used in MagLev. For such antibody test solutions, binding of the antibodies to the antigens bound on the surface of the substrates can be carried out outside of the DeLISA equipment, the bound substrates recovered and introduced into the subsequent density amplification environment and/or the MagLev equipment.

In certain alternative embodiments, if detection of antigens in a test solution is desired, antibodies can be bound onto the surface of a substrate. Binding of specific antibodies onto the surface of a substrate can be carried out using various different techniques that would be readily apparent to one of ordinary skill in the art, can also be utilized.

### Density Amplification

Many different density amplification schemes can be utilized. In certain embodiments, density change can be amplified by the growth of a density amplification material, such as metals or polymers, on the substrate that has formed an antigen-antibody complex. The density amplification material preferably has a density that differs from the substrate and both of the antigen and antibody. Some suitable density amplification material include gold, silver, iron, mercury, nickel, copper, platinum, palladium, cobalt, iridium ions, polymer and the like, including mixtures of such density amplification materials.

In certain embodiments, gold can be grown as described in the examples below.

In certain embodiments, silver can be grown as described in the examples below.

In certain embodiments, acrylate polymers can be grown as described in Sikes et al., “Antigen detection using polymerization-based amplification,” Lab on a Chip, vol. 9, no. 5 (March 2009) pp. 653-656, the specific contents regarding polymerization of the acrylate polymers being incorporated by reference herein. Here, the substrate can be a different polymer so that the growth of the acrylate polymer, which has a different density, can lead to a density amplification. The density amplification can progress as long as desired (to obtain a measurable change in the levitation height) so that even small density differences between the antigen-antibody complexed substrate and the acrylate (or any other density amplification) polymer can be amplified over time.

According to one or more embodiments, after complexation of an antibody to a substrate antigen (or vice versa), the density of the complex in increased (or decreased) by growth of a density amplification material on the surface of the substrate. The density amplification material has a density that is different from that of the substrate antigen-antibody complex. In some embodiments, the density amplification material is of a lower density than the substrate antigen-antibody complex, e.g., it is a low density organic polymer. In some embodiments, the density amplification material is of a higher density than the substrate antigen-antibody complex, e.g., it is a high density metal. Growth of the density amplification material on the substrate results in a detectable change in density of the suspended substrate.

In certain embodiments, density amplification can be carried in multiple steps. In certain embodiments, secondary antibodies can be utilized, which can be attached to the antibodies to be detected in the test solution. The secondary antibodies can contain density amplifying materials, such as gold-labels or silver-labels, which can catalyze the growth of the density amplification material onto the surface of the substrates to increase the density.

In certain embodiment, the density amplification can be carried out in the presence of a catalyst. For instance, the bound antibodies may be provided selectively with a catalyst that enhances the addition or growth of the density amplification material. For example, secondary antibodies containing catalysts can be utilized to grow gold or silver. Some suitable catalysts include gold, silver, platinum, lead, transition metals, enzymes such as horseradish peroxidase, alkaline phosphatase, glucose oxidase, and the like. Soluble metal salts, such as Au³⁺, Ag²⁺, and the like can be reduced by reducing agents, such as hydroxylamine, hydroquinone and the like to produce elemental metal. Catalysts, such as gold nanoparticles, adsorbed to biomolecules serves to accelerate the reduction of soluble salts into insoluble elemental metal that deposits specifically only when the relevant biomolecular binding events have taken place on the bead.

In certain embodiments, density amplification is an attachment of a fixed sized object, such as a pre-formed nanoparticle onto the surface of the substrate. In other embodiments, density amplification can involve the growth of an density amplification material, where the growth, and consequently density change, can be tuned as desired.

In certain embodiments, growth of the density amplification material may be carried out to achieve a minimum change in the levitation height (relative to the untreated conjugate), to achieve satisfaction of a targeted sample treatment time, to achieve differences in the type of antibodies detected, and the like.

The change in density of a substrate due to the deposition of an arbitrary coating, expressed in terms of the specific density and volume of the coating material, ρ_{coating}, V_{coding}, and the specific density and volume of the object ρ_{substrate}, V_{substrate} is given by Eq. 1.

$$Δρ = \frac{{ρ}_{coating} - {ρ}_{substrate}}{( 1 + ( \frac{{V}_{substrate}}{{V}_{coating}} ) )} \qquad ( 1 )$$

For the same volume of coating material, a larger change in density of the substrate can be obtained by increasing the difference between ρ_{coating} and ρ_{substrate}, or by increasing the surface area to volume ratio of the substrate.

One particular example can demonstrate the importance of the density amplification material. Typically diamagnetic substrates include colloidal microparticles from organic polymers such as polystyrene (PS), polymethylmethacrylate (PMMA), agarose, nylon, paper, nitrocellulose, Immunodyne, or any other polymers that are about 600 microns in size. Typical metal nanoparticles include gold nanoparticles that are about tens of nanometers in size. If 10 nm diameter gold nanoparticles are attached to a surface with a hexagonal close packing, approximately 8×10⁹ nanoparticles can fit on the surface. This level of adsorption causes a 0.0007 g/cm³ change in the density of the microparticles, which is not reliably detectable as a levitation height difference in a MagLev environment. Moreover, this assumes a very tight hexagonal close packed gold nanoparticles, which is not likely to occur in real materials. More typically, a relatively dilute serum samples or other biological samples are used and the amount of gold nanoparticles that are adsorbed on the surface can be as low as 5×10⁴ to 5×10⁹ nanoparticles. Such low amount of nanoparticles can result in density changes that are too small to be detected by MagLev.

According to one or more embodiments, density amplification is carried out by growing gold onto the beads. Assuming a growth rate of 0.1 nm/s, after 30 minutes of amplification, the nanoparticle grows to about 370 nm in diameter causing the density to change by ˜0.037 g/cm³ which is well within the range of detection in MagLev.

FIG. 1D shows the evolution of the final density of beads after a given amount of amplification time. Longer amplification times lead to greater changes in density and provides the ability to detect lower concentrations of surface bound nanoparticles.

### EXAMPLES

The following examples demonstrate that DeLISAs performed using non-porous spherical polystyrene beads (PS beads) as a solid substrate can be used to detect clinically relevant concentrations of antibodies (ng/ml) against HIV1, hepatitis C and syphilis in a model liquid sample (phosphate buffered saline (PBS) with 0.5% bovine serum albumin (BSA). In some embodiments results were obtained within 30-40 minutes by carrying out a reaction on a non-porous bead. The below examples are not meant to be limiting or exclusive.

### Example 1

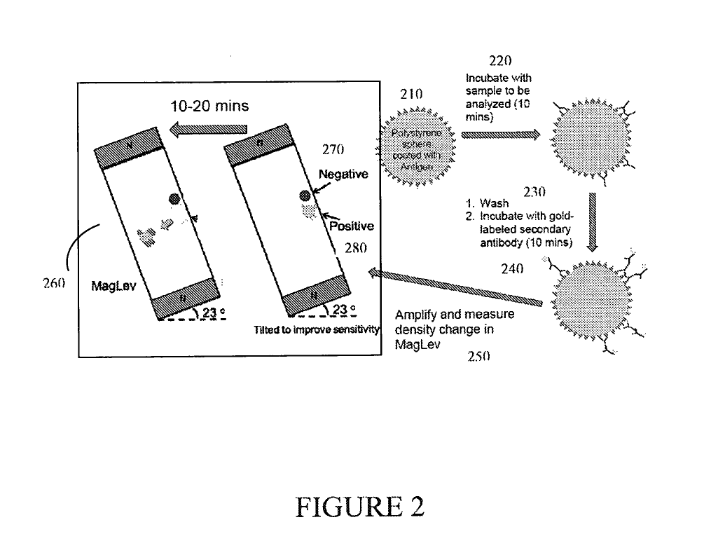

As shown in FIG. 2, one embodiment of the DeLISA assay procedure involves coating polystyrene spheres with an antigen 210, incubating the sphere with the sample to be analyzed for 10 minutes 220, washing 230, incubating the spheres with gold-labeled secondary antibodies for 10 minutes 240, amplifying and measuring the density changes in MagLev 260. The MagLev system 260 is used to separate (in 10 to 20 minutes) a negative control sphere 270 from a positive sphere 280 which detects the sample and experiences a change in density.

### Example 2

The DeLISA assay procedure was performed using non-porous polystyrene beads (PS beads).

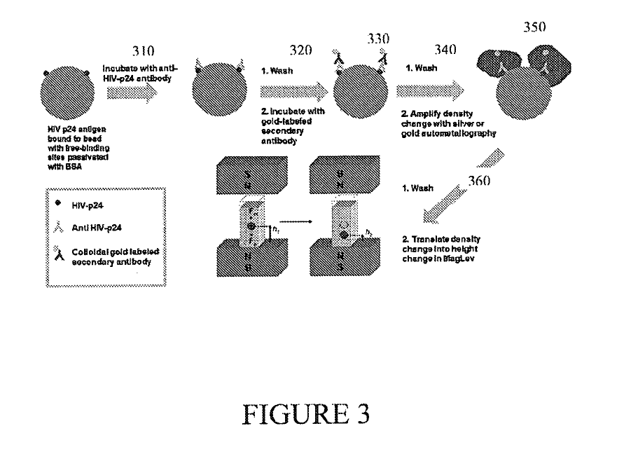

The preparation and execution of a this procedure is shown in FIG. 3. p24-coated PS beads were prepared by soaking clean PS beads in a solution of p24 310. These beads were rinsed thoroughly and can be stored for extended periods of time (at least one week) without any apparent decrease in the amount of adsorbed p24. The batches of ˜30 p24-coated PS beads were transferred to vials containing goat anti HIV-1 p24 antibody dissolved in a model liquid (0.5% BSA in PBS). Using 10-fold serial dilutions, the concentration of anti-p24 antibody was varied from 200 nM to 2 pM. After incubating the beads in these solutions of antibody for four hours, they were rinsed 320 and added to a solution of colloidal gold-labeled anti-goat IgG antibody 330. A subsequent forty minute incubation was followed by several rinsing steps 340. In order to increase the change in the density of the beads, they were subjected to a gold-catalyzed silver density amplification reaction 350. These changes in density were detected by levitating the beads in a solution of 0.3 M MnCl₂ placed within a typical MagLev device 360.

More specifically, polystyrene (PS) heads (250-750 μm) were rinsed thoroughly with acetone followed by phosphate buffered saline (PBS, 0.25 M). HIV-1 p24 antigen was adsorbed to these beads by incubating them overnight at 4° C. in a solution of HIV-1 p24 (200 μg/ml) dissolved in PBS (0.25 M). Unbound p24 was removed by rinsing the PS beads with buffer A (0.5% w/v BSA and 0.1% w/v NaN₃ dissolved in 0.25 M PBS) (3 ×5 min). The antigen-coated beads can be stored in buffer A at 4° C. for at least one week with no apparent decrease in the concentration of adsorbed p24 protein. Batches of ˜30 p24-coated PS heads were transferred to vials containing goat anti HIV-1 p24 antibody dissolved in buffer A. Using 10-fold serial dilutions, the concentration of anti-p24 antibody was varied from 200 nM to 2 pM. After incubation for four hours, the beads were rinsed with buffer A (3 ×5 min) before adding a solution of anti-goat IgG conjugated to 10 nm diameter colloidal gold (1:250 dilution in buffer A). The beads were allowed to incubate with the secondary antibody for 40 minutes, after which they were rinsed with buffer A (3 ×5 min), PBS (0.25 M, 2×5 min), and DI water (2×5 min). The beads were then treated with either gold or silver density amplification solution for 25 minutes. The density amplification reactions were quenched by thorough rinsing with DI water. [Silver density amplification solution cost ˜$0.25 per mL, gold density amplification solution cost ˜$3 per mL. Typically 0.1 nil per assay is used.] The change in the density of the beads was assessed by levitation in an aqueous solution composed of 0.3 M MnCl₂ and 0.2 M ZnBr₂.

Specific recognition and binding of anti-HIV-1 p24 antibodies from the liquid sample to HIV-1 p24 antigens immobilized on the PS beads did not cause detectable changes in the density of the beads. Antigen-antibody binding events are amplified to cause detectable changes in density by employing silver or gold autometallography, catalyzed by a colloidal gold-linked secondary antibody.

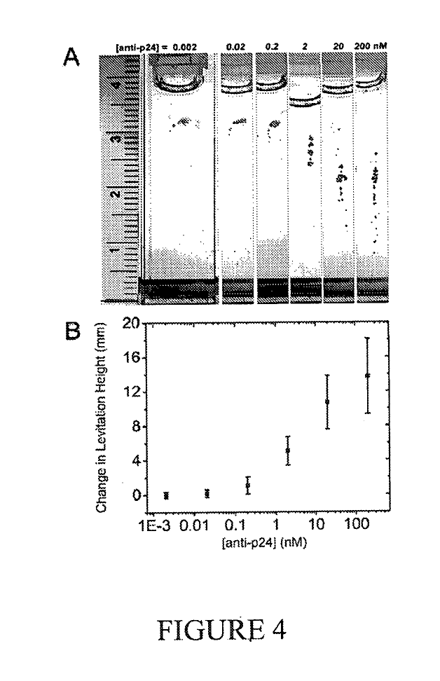

FIG. 4A shows the results of the immunoassay using silver density amplification on spherical PS beads. MagLev allows the density of a bead to be directly correlated with its levitation height. The lower the levitation height of a bead, the higher its density. As shown, an increase in the density of a bead occurs when the bead is coated with silver in the autometallography step. Beads exposed to a liquid sample with a higher concentration of anti-HIV-1 p24 antibody have, on average, lower levitation heights in a MagLev device. The mean levitation height of the beads after the amplification step is proportional to the concentration of anti-HIV-1 p24 antibodies that was present in the model liquid samples. These results, therefore, suggest that the change in the density of a bead provides a quantitative readout of the concentration of anti-HIV-1 p24 antibody in a liquid sample.

FIG. 4B shows the average levitation height of the heads subjected to each concentration of anti-HIV-1 p24 antibody. In certain embodiments, the immunoassays described herein may be sensitive enough to provide detection in even nanomolar (and higher) ranges.

The spread in levitation heights of beads treated identically may be due to a number of factors, including the polydispersity of the beads, heterogeneous coating of the beads with the gold-labeled antibodies, and/or the autocatalytic nature of autometallography. In certain embodiments, spread in levitation heights might be mitigated by using monodisperse PS beads or with other solid supports, or by using one large head rather than a collection of small beads.

In this assay, it was possible to unambiguously detect the presence of 2 nM of anti-HIV p24 antibodies in a liquid sample. This example demonstrates that the DeLISA assay procedure can be used to quantify the concentration of anti-HIV-1 p24 antibodies in a model liquid sample (phosphate buffered saline (PBS) with 0.5% bovine serum albumin (BSA)).

Materials: Manganese (II) chloride tetrahydrate (ACS reagent grade, >98%), Zinc bromide (puriss., anhydrous, ≧98%), Anti-Goat IgG (whole molecule)—Gold antibody produced in rabbit (affinity isolated antibody, aqueous glycerol suspension, 10 nm (colloidal gold)) and silver density amplification kits were purchased from Sigma-Aldrich. Gold density amplification solution was purchased from Nanoprobes. Anti HIV-1 p24 antibody (goat) and HIV-1 p24 were purchased from Abeam. Polystyrene beads crosslinked with divinylbenzene (250-750 μm diameter) were purchased from Polysciences Inc. Kapton™ sheets were purchased from McMaster Carr.

### Example 3

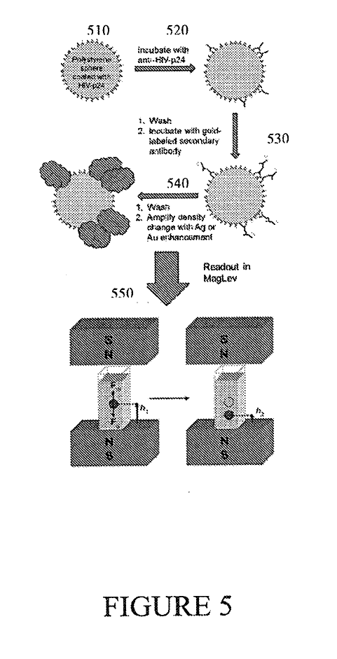

The preparation and execution of a DeLISA is shown in FIG. 5. The p24-coated PS beads were prepared by soaking clean PS beads in a solution of p24, 510. These beads were rinsed thoroughly and can be stored for extended periods of time (at least one week) without any apparent decrease in the amount of adsorbed p24. The batches of ˜30 p24-coated PS beads were transferred to vials containing goat anti HIV-1 p24 antibody dissolved in a model liquid (0.5% BSA in PBS). Using 10-fold serial dilutions, the concentration of anti-p24 antibody was varied from 200 nM to 2 pM. After incubating the beads in these solutions of antibody for four hours, 520, they were rinsed and a solution of colloidal gold-labeled anti-goat IgG antibody raised in rabbit was added, 530. A subsequent forty minute incubation was followed by several rinsing steps. At this stage, no changes in the density of the PS beads using MagLev could be detected.

In order to increase the change in the density of the beads, they were subjected to a gold-catalyzed silver density amplification reaction, 540. These changes in density were detected by levitating the beads in a solution of 0.3 M MnCl₂ placed within a typical MagLev device, 550.

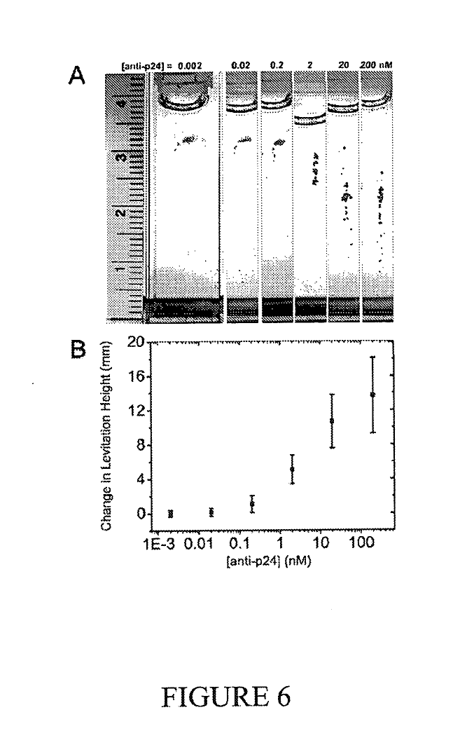

FIG. 6A shows the results of the immunoassay using gold-catalyzed silver density amplification on spherical PS beads. MagLev allows the density of a bead to be directly correlated with its levitation height. The lower the levitation height of a bead, the higher its density. As shown, an increase in the density of a bead occurs when the bead is coated with in the electroless deposition step. Beads exposed to a liquid sample with a higher concentration of p24 antibody have, on average, lower levitation heights in a MagLev device than beads exposed to a liquid sample with a lower concentration of anti-HIV-1 p24 antibody. These results, therefore, suggest that the change in the density of a bead provides a quantitative readout of the concentration of anti-HIV-1 p24 antibody in a liquid sample.

FIG. 6B shows the average levitation height of the beads subjected to each concentration of anti-HIV-1 p24 antibody. The error bars indicate one standard deviation in the mean, calculated from a single experiment. The spread in levitation heights of beads treated identically may be due to a number of factors, including the polydispersity of the beads, heterogeneous coating of the beads with the gold-labeled antibodies, and/or the autocatalytic nature of autometallography.

This example demonstrates that. DeLISA unambiguously detects the presence of 2 nM of anti-HIV p24 antibodies in a liquid sample. This concentration is within the clinically relevant range for HIV immunoassays.

### Example 4

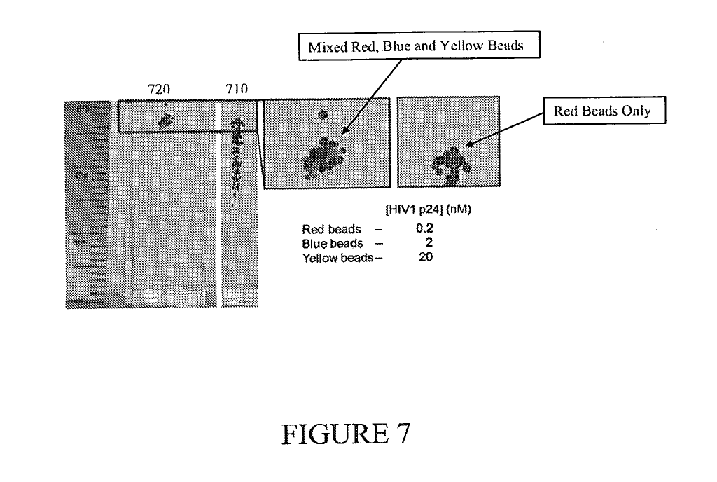

FIG. 7 shows photographs of a quantitative singleplex (i.e. single antibody type) DeLISA assay for HIV1 antibodies in serum, according to an embodiment of the present disclosure. The colored beads were exposed to samples of varying concentration of HIV-1 antibodies and silver amplified for 18 minutes. Beads exposed to samples containing 2 and 20 nM HIV1 change levitation heights, 710, while beads exposed to 0.2 nM do not, 720. This demonstrates that information regarding, different concentrations of even the same target can even be obtained.

### Example 5

A related embodiment of singleplex assay has beads exposed to serum antibody with the clinically relevant concentration for active HIV infection. The beads are treated so that the red beads are the negative control, the blue beads are the positive control and the yellow beads assay a patient's serum. In the DeLISA assay, if the red beads sink, then the assay failed. If the blue beads do not sink then the assay failed. If the yellow beads sink, then the patient is HIV positive. If the yellow beads remain at the same levitation height as the red beds then the patient is HIV negative.

### Example 6

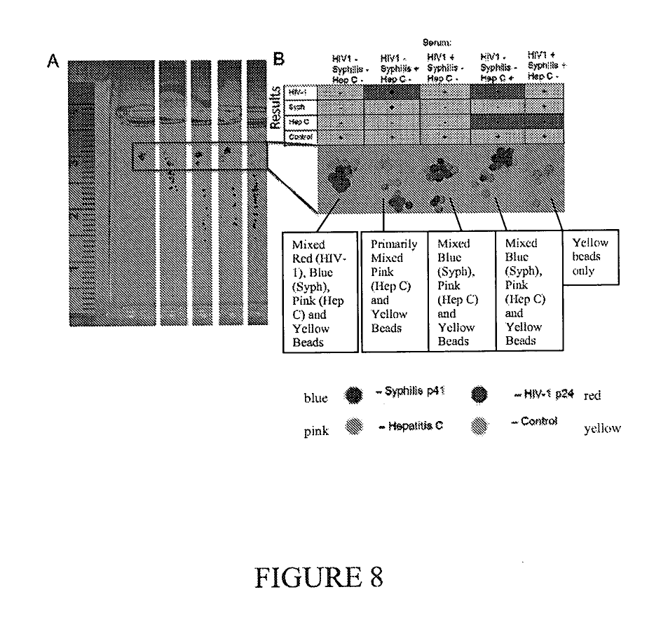

FIG. 8A-B show results from a multiplex DeLISA assay. 600 μm colored polystyrene beads were adsorbed with different antigens. Blue beads had p4.1 protein which is diagnostic for syphilis, pink beads had hepatitis C, red heads had HIV p24 and yellow beads were free of antigens. Model serum samples were prepared by doping goat serum with purified goat antibodies against the disease targets. Beads were exposed to these test serums and then developed with a DeLISA. FIG. 8A shows that antibodies against the antigens if present in the serum sample adsorb onto the beads. Silver amplification detects and amplifies these binding events by changing the density of the bead. FIG. 8B shows a table of how the assay (with the corresponding serum conditions as described above each column) was read by observing which beads did not change levitation heights. Thus the presence of a colored bead in the readout section (inset from FIG. 8A shown below table) indicates a serum sample is negative for the particular antibody. Yellow beads, which are the negative controls should always be it the readout section. False results are colored dark. HIV-p24 seems to react under all conditions. Hepatitis C does riot seem to react. Control beads do not react.

Materials for Examples 2-5: Manganese (II) chloride tetrahydrate (ACS reagent grade, >98%), Zinc bromide (puriss., anhydrous, ≧98%), Anti-Goat IgG (whole molecule)—Gold antibody produced in rabbit (affinity isolated antibody, aqueous glycerol suspension, 10 nm (colloidal gold)) and silver density amplification kits were purchased from Sigma-Aldrich. Gold density amplification solution was purchased from Nanoprobes. Anti HIV-1 p24 antibody (goat) and HIV-1 p24 were purchased from Abeam. Polystyrene beads crosslinked with divinylbenzene (500-600 μm diameter) were purchased from Polysciences Inc. Kapton™ sheets were purchased from McMaster Can.

### Procedure for Dyeing the Beads for Examples 4-6:

4 mg of a dye (Sudan Red, Reactive Blue, Alazarin Yellow) was dissolved in 1.5 mL of 10:1 toluene:ethanol. These solutions were then passed through cotton filters to remove any remaining particulates. Polystyrene beads (˜100 mg) were then added, and the solutions were gently rocked for one hour. After incubation, the beads were thoroughly rinsed with ethanol and dried in vacuo for 4 hours.

### Preparation of Antigen-Coated Beads for Examples 3-6:

Polystyrene (PS) beads (650 μm) were rinsed thoroughly with ethanol followed by phosphate buffered saline (PBS, 0.25 M). Antigens on these beads were adsorbed by incubating them overnight at 4° C. in a solution of antigen (100 μg/ml) dissolved in PBS (0.25 M). Unbound antigen was removed by rinsing the PS beads with buffer A (0.5% w/v BSA and 0.1% w/v NaN₃ dissolved in 0.25 M PBS) (3 ×5 min). The antigen-coated heads can be stored in buffer A at 4° C. for at least two weeks with no apparent decrease in the concentration of adsorbed antigen.

### Detailed DeLISA Procedure for Examples 3-6:

Batches of ˜5-10 antigen-coated PS beads were transferred to vials containing the appropriate goat anti-antigen antibody dissolved in buffer A. After incubation for four hours, the beads were rinsed with buffer A (3 ×5 min) before adding a solution of anti-goat IgG conjugated to 0.10 nm diameter colloidal gold (1:250 dilution in buffer A). The beads were allowed to incubate with the secondary antibody for one hour, after which time they were rinsed with buffer A (2×5 min), PBS (0.25 M, 3 ×5 min), and DI water (3 ×5 min). The beads were then treated with freshly prepared silver density amplification solution for 25 minutes. The density amplification reactions were quenched by transferring the beads to a solution of 0.25% w/v sodium thiosulfate in water. The change in the density of the beads was assessed by levitation in an aqueous solution composed of 0.3 M MnCl₂ and 0.15 M ZnBr₂.

### Example 7

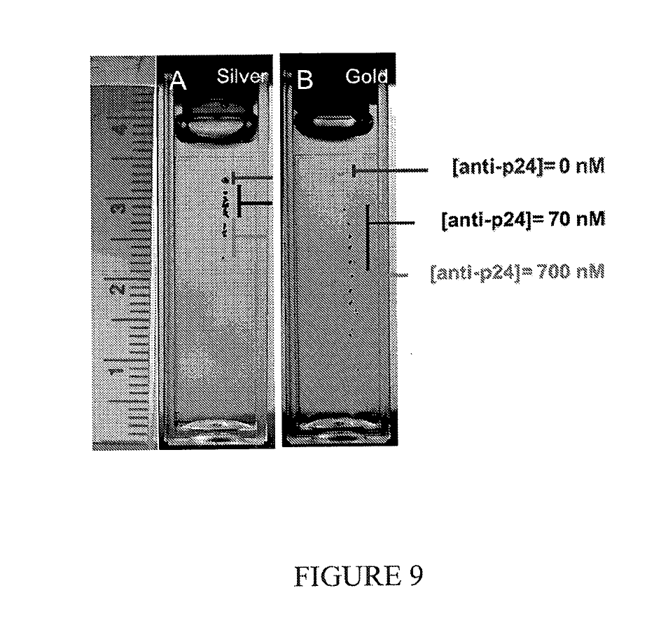

FIG. 9 A-B show a comparison between silver and gold autometallography. PS beads were prepared for anti-HIV-1 p24 antibody DeLISA as detailed in Example 1. As shown in FIG. 9 A-B both gold and silver amplification result in a detectable change in levitation height of beads exposed to a liquid sample containing anti-HIV-1 p24 antibodies. In FIG. 9A the amplification step was performed with silver, while in FIG. 9B the amplification step was performed with gold. It is apparent that gold density amplification leads to a larger change in the levitation height. The large spread in the beads post density amplification causes beads exposed to different antibody concentrations to have partially overlapping ranges in levitation heights. Gold amplification results in larger changes in levitation heights than silver amplification. While not wishing to be bound by any specific theory, this difference in levitation heights can be rationalized by recognizing that the specific density of gold (19.6 g/cm³) is almost twice that of silver (10.6 g/cm³).

### Example 8

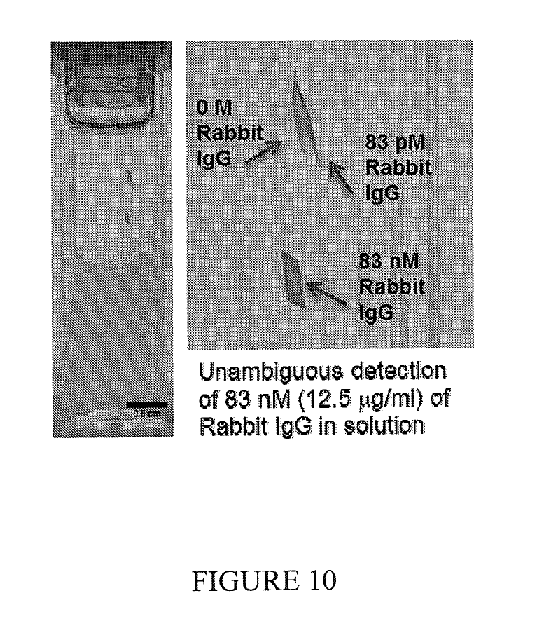

The present disclosure is not limited to utilizing spherical PS particles FIG. 10 shows a photograph (Scale bar is 500 mm) of Kapton sheets used in a DeLISA assay, according to an embodiment of the present disclosure. Katon sheets were prepared for anti-HIV-1 p24 antibody DeLISA in the same manner as PS beads, as detailed in Example 1. This experiment shows results using Kapton sheets in place of spherical PS spheres (which have the smallest surface area for a given volume). For similar amplification times, substrates with larger surface area to volume ratios show greater density changes. Other types of particle shapes and materials may be utilized.

### Example 9

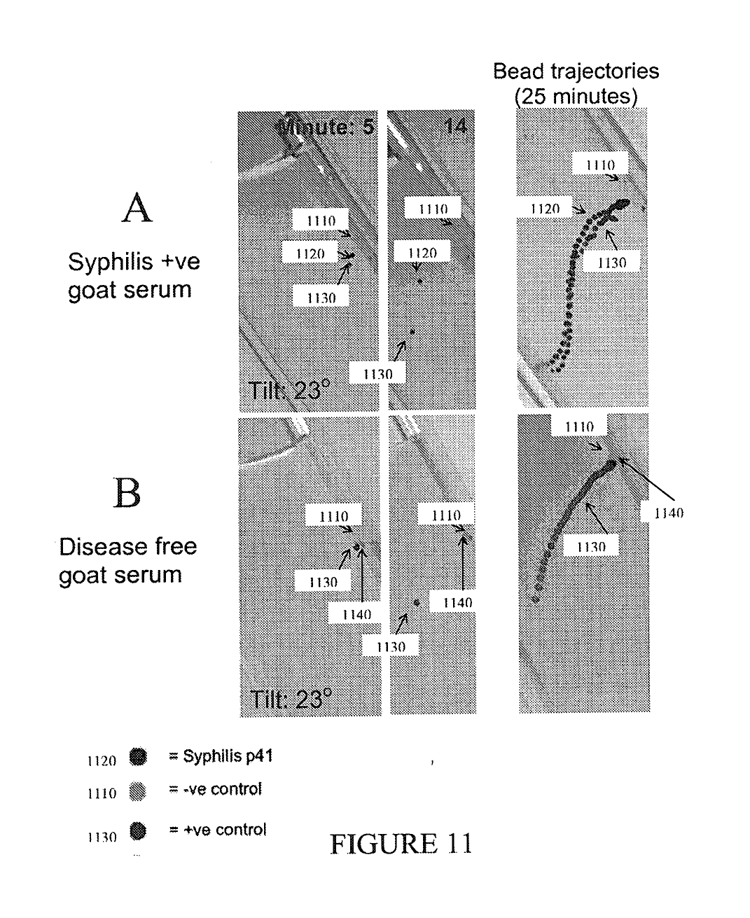

FIG. 11A-B shows a DeLISA for Syphilis at 5, 14, and 25 minutes. PS beads were prepared for Syphilis DeLISA as detailed in Example 1 (except with Syphilis antigens and antibodies in place of HIV p24). In FIG. 11A, the −ve control beads 1110, the Syphilis p41 heads 1120, and the +ve control beads 1130 are shown. The +ve control and Syphilis p41 beads moved during the experiment, while the −ve control beads did not.

In FIG. 11B, the −ve control beads 1110, the disease free goat serum beads 1140, and the +ve control beads 1130 are shown. Only the +ve control beads moved during the experiment, while the −ve control beads 1110, the disease free goat serum heads 1140 did not. The total assay time was 40 minutes.

This example demonstrates that DeLISA can be used to detect the presence of Syphilis. Beads which were positive for Syphilis (1120) could be separated in the Mag Lev device from beads without the Syphilis bacteria (1110). However, beads which did not have the Syphilis bacterium (1140), did not experience a change in density due to antibody binding and were not separated from the control particles (1110).

### Example 10

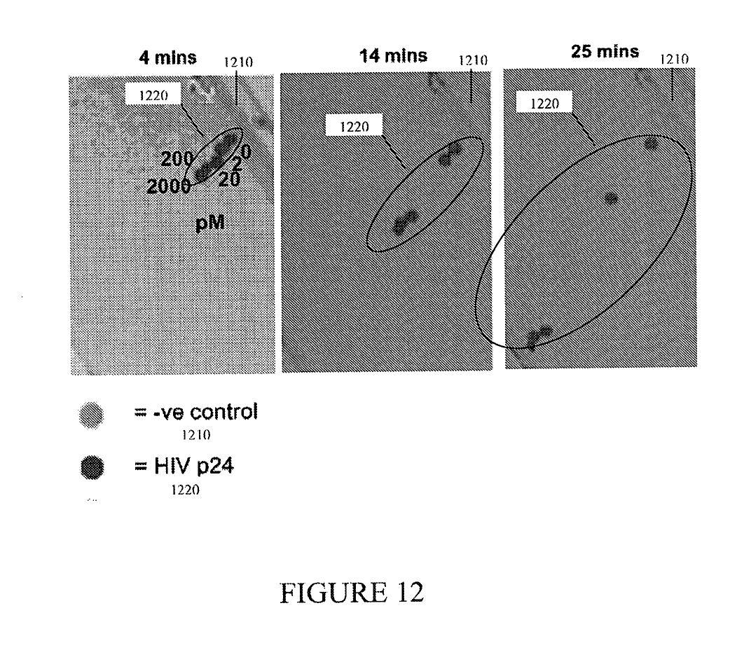

FIG. 12 shows a quantitative DeLISA for HIV antibodies. PS beads were prepared for anti-HIV-1 p24 antibody DeLISA as detailed in Example 1. The −ve control bead 1210 does not move during the experiment. HIV p24 beads 1220 of different concentrations (pM, as labeled on the image) arc circled for clarity. The system is shown at 4, 14, and 25 minutes. The beads were incubated for 2 hours with goat anti-HIV-1 IgG.

This example demonstrates detection of 2 pM of HIV antibody. Beads which were positive for HIV (1220) could be separated with DeLISA in the Mag Lev device from beads without HIV (1210). The beads which did not have HIV (1210), did not experience a change in density due to antibody binding and did not move in the experiment.

### Example 11

In order to study of discrete reactions in droplets, a single bead in a single capillary procedure was carried out that allowed the observation of the changes of density of single heads in individualized reaction chambers with limiting concentrations of reagents.

Manganese (II) sulfate monohydrate (USP grade), Anti-Goat IgG (whole molecule)—Gold antibody produced in rabbit (affinity isolated antibody, aqueous glycerol suspension, 10 nm (colloidal gold)), and silver density amplification solution were purchased from Sigma-Aldrich. Anti HIV-1 p24 antibody (goat) and HIV-1 p24 were purchased from Abeam. Polystyrene beads (PS) crosslinked with divinylbenzene (500-600 μm diameter) were purchased from Polysciences Inc.

4 mg of dye, either Sudan Red, Reactive Blue, Alazarin Yellow, Fat Brown or Solvent Green, was dissolved in 1.5 mL of 10:1 toluene:ethanol. After 10 minutes the dye solutions were passed through cotton filters to remove any remaining particulates. Approximately 100 mg of PS beads were then added into the dye solution and gently rocked for one hour. The beads were then thoroughly rinsed with ethanol and dried in vacuo for at least 4 hours (typically overnight).

The dried dyed PS beads were rinsed thoroughly with ethanol followed by phosphate buffered saline (PBS, 0.25 M).

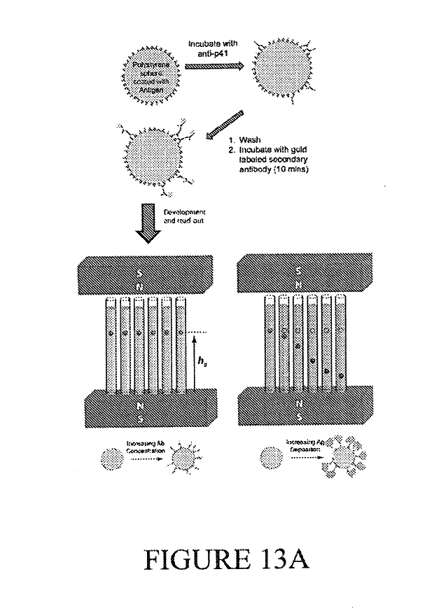

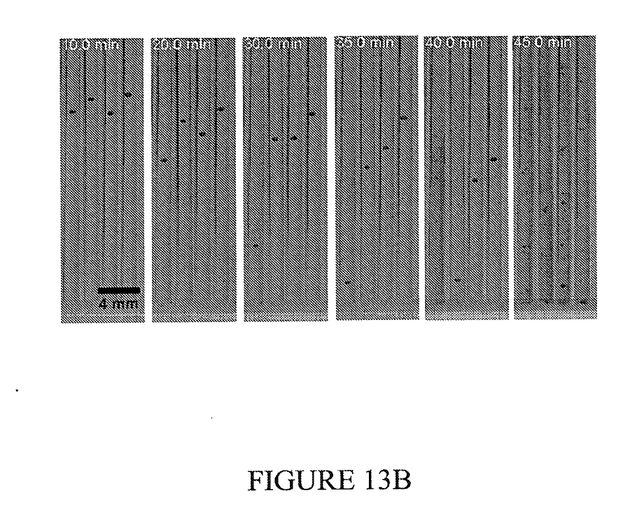

As shown in FIG. 13A, antigens of interest were adsorbed onto the surface of colored spherical polystyrene beads (600 μm in diameter). Specifically, antigens were adsorbed on the colored beads by incubating them overnight at 4° C. in a solution of antigen (100 μg/ml) dissolved in PBS (0.25 M). Unbound antigen was removed by rinsing the PS beads with buffer A (0.5% w/v BSA and 0.1% w/v NaN3 dissolved in 0.25 M PBS) (3 ×5 min). The antigen-coated beads can be stored in buffer A at 4° C. for at least two weeks with no apparent decrease in the concentration of adsorbed antigen.

The immunosorbed beads were then incubated in liquid samples for a total of 10 minutes. After incubation for 10 minutes, the beads were removed and rinsed in fresh 200 μl buffer A by pipetting gently 10 times before being transferred into a solution of anti-goat IgG conjugated to 10 nm diameter colloidal gold (1:5 dilution in PBS). The washing was carried out remove non-specifically bound antibodies. The beads were allowed to incubate for 10 minutes, after which they were rinsed with.

Then, the beads were incubated in a solution containing gold-labeled secondary antibodies for 10 minutes. The beads were wash again in buffer A (1×), and deionized water (3×) to remove non-specifically bound secondary antibodies and to remove traces of chloride ions from the buffer, which could interfere with the silver amplification process. The beads were now ready for amplification and readout.

Beads were loaded into glass capillaries containing 300 mM MnSO₄ (paramagnetic ion for MagLev) dissolved in a commercial silver density amplification solution (Sigma). To prepare the glass capillaries, horosilicate glass melting point capillaries (Kimble-Chase) with inner diameters ranging from 0.8-1.1 mm were cut to a height of 4.5 mm. The capillaries were washed with a micellar solution of SDS (50 microliters of the solution were pipetted in and out) and then dried.

Up to 7 antigen-coated PS beads were transferred to vials containing the appropriate goat anti-antigen antibody dissolved in buffer A and only one bead was loaded per capillary. The capillaries were pre-wetted with an 8.3 mM (which is the critical micelle concentration (cmc)) aqueous solution of sodium dodecyl sulfate and dried prior to loading with the silver solution. This pre-treatment prevented the sticking of the beads to the capillary walls. Approximately 50 μl of silver containing solution were added to each capillary. The capillaries containing the beads were then sealed with tacky wax (Bard's) and the capillaries were placed upright in a custom-built plastic holder before loading into a MagLev device. The evolution of the levitation height of the beads were monitored through time-lapse photography.

FIG. 13B shows time-lapse images of a successful DeLISA against syphilis in goat serum. From left to right each capillary was loaded with beads exposed to 1:10, 1:100 and 1:1000 dilutions of syphilis positive goat serum. The right most bead is a control.

As shown, at the beginning of the assay, all the beads (each previously incubated in 1:10, 1:100 or 1:1000 dilutions of syphilis positive goat serum, or incubated in a control sample that did not have any antibodies) levitate at the height that corresponds to the density of the unmodified polystyrene beads. It is apparent that the beads show variations in their starting densities.

Over time, the beads decreased in levitation height and changed color from blue to gray. Beads exposed to samples with lower dilutions of the serum (or higher concentrations of serum) sink the fastest, while the control beads were the last to sink.

At longer times, the solution starts turning brown due to precipitation of silver and large deposits of silver form and sink to the bottom of the capillary.

After about 45 minutes all beads, including the control beads, sink to the bottom of the capillary.

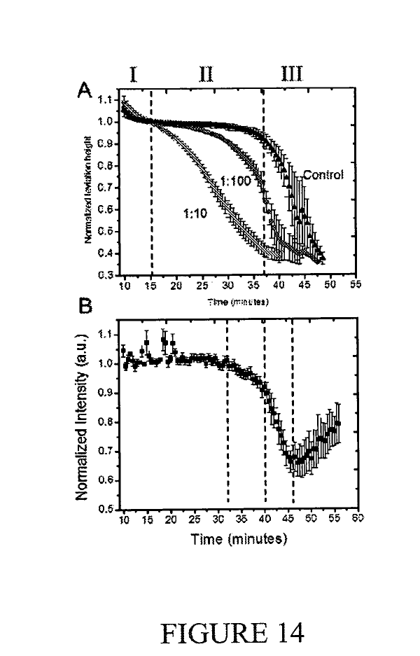

To characterize quantitatively the evolution of the DeLISA, data from four beads prepared identically for each dilution of syphilis positive serum were averaged and the levitation height was plotted as a function of time (see FIG. 14A).

The levitation height was normalized by dividing the height at each time point with the levitation height at 15 minutes to account for the different initial densities.

Three regions can be identified on the curves. The equilibration region, during which the beads moved to a stable levitation height, lasted about five minutes. After 5 minutes, the evolution of the levitation height of the beads depended on the concentration of antibodies that was present in the sample. Beads incubated in samples containing 1:10 dilution of the syphilis positive serum started sinking at 15 minutes and reached the bottom of the capillary after about 40 minutes. Beads incubated in samples containing 1:100 dilution of the syphilis positive serum started sinking ˜25 minutes and reached the bottom of the capillary at about 45 minutes. The control beads in contrast started sinking after 35 minutes and reached the bottom of the capillary ˜50 minutes.

The four replicate beads all show very similar trajectories up to 35 minutes, after which their trajectories become noisy (i.e. the error bars become larger).

The levitation height, h, of a substrate of density, ρₘ, with a magnetic susceptibility of χₘ in a solution of density, ρₛ, with magnetic susceptibility, χₛ, in a magnetic levitation device with magnets of strength B₀ on its surface separated by distance d is given by equation 2. μ₀ is the permeability of free space and g is the gravitational constant.

$$h = \frac{( {ρ}_{s} - {ρ}_{m} ) g {μ}_{o} {d}^{2}}{( {x}_{s} - {x}_{m} ) 4 {B}_{0}^{2}} + \frac{d}{2} \qquad ( 2 )$$

In a DeLISA a change in the levitation height of the beads occurs due to two reasons: (i) ρₘ increases due to the deposition of silver onto the particles, (ii) ρₛ decreases slightly due to silver depositing specifically onto the particles, and massively due to silver metal precipitating out of solution and settling to the bottom of the capillaries, the latter occurring at much longer time scales.

To obtain an indication of the rate of the decrease in pₛ due to the precipitation of silver, the grayscale intensity of the solution in the capillary was measured (choosing representative regions above the beads) from the photograph images. The intensity of all 12 capillaries were averaged and the results are shown in FIG. 14B. The intensity of the solution decreased only slightly in 35 minutes, after which the intensity changed rapidly which indicated large-scale precipitation of silver. After 45 minutes the intensity of the solution increased since large silver particles that formed sedimented to the bottom of the capillary.

The region of rapid silver precipitation corresponded to the region where all beads sink rapidly to the bottom of the capillary.

Without wishing to be bound by theory, it is believed that the sinking of the beads at times less than 35 minutes is due predominantly to an increase in ρₘ, and at longer times due to the decrease in ρₛ.

### Advantages

DeLISA provides an immunoassay that is quantitative, easy to multiplex and does not require complex equipment or electricity for readout. Particularly, DeLISA can be easily multiplexed to detect multiple analytes since several beads can be placed in single serum sample to detect, such as HIV, syphilis and Hepatitis C simultaneously.

DeLISA can be used to detect clinically relevant concentrations of antibodies (ng/ml) against HIV1, hepatitis C and syphilis in a model liquid sample (phosphate buffered saline (PBS) with 0.5% bovine serum albumin (BSA).

Moreover, in certain embodiments, the rate of change of the levitation height of the beads can correlate with the amount of antibody that was present in the liquid sample, thus allowing quantitation by measurement of the levitation height.

Moreover, DeLISA can be carried out rapidly. For example, the time to conduct the antibody binding steps can be carried within about half an hour (e.g., 25 minutes), while readout of the assay can be conducted 20-35 minutes after loading into the MagLev device.

While there have been shown and described examples of DeLISA, it will be readily apparent to those skilled in the art that various changes and modifications may be made therein without departing from the scope of the invention.

1. A method for detecting binding of target molecules comprising:
providing a diamagnetic substrate comprising one or more target binding molecules to obtain an target binding molecules-attached substrate;
contacting the target binding molecules-attached substrate to a sample containing target molecules that selectively attach to said one or more target binding molecules to obtain target-bound substrate;
altering the density of the target-bound substrate to obtain a density-amplified substrate;
introducing said density-amplified substrate into a paramagnetic liquid;
applying a magnetic field to the paramagnetic liquid, wherein the density-amplified substrate attains a levitation height, said levitation height being different from the levitation height of the target-bound substrate, target binding molecules-attached substrate, or diamagnetic substrate under the same magnetic levitation conditions.

2. The method of claim 1, wherein said target binding molecules are antigens and said target molecules are antibodies or antibody fragments.

3. The method of claim 1, wherein said target binding molecules are antibodies or antibody fragments and said target molecules are antigens.

4. The method of claim 1, wherein said target binding molecules are nucleic acids and said target molecules are complementary nucleic acids.

5. The method of claim 1, wherein said target binding molecules are proteins and said target molecules are complementary proteins.

6. The method of claim 2, wherein said antigens include HIV p24 antigen, Syphilis p41 antigen, Hepatitis Core antigen, haptens of antibiotics, haptens of penicillin, haptens of ampicillin, haptens of chloramphenicol or combinations thereof.

7. The method of claim 2, wherein said antibodies include HIV, Syphilis, Hepatitis C, rubella, penicillin, ampicillin, chloramphenicol, or combinations thereof.

8. The method of claim 1, wherein altering the density of the target-bound substrate includes growing gold onto the target-bound substrate.

9. The method of claim 8, wherein said growing gold onto the target-bound substrate includes providing secondary moieties that attach to the target-bound substrate, where the secondary moieties have a catalyst that provide catalytic growth of gold.

10. The method of claim 1, wherein said amplifying density change includes growing silver onto the target-bound substrate.

11. The method of claim 10, wherein said growing silver onto the target-bound substrate includes providing secondary moieties that attach to the target-bound substrate, where the secondary moieties have a catalyst that provide catalytic growth of silver.

12. The method of claim 1, wherein said sample comprises less than 200 nM of target molecules.

13. The method of claim 1, wherein said sample comprises less than 2 nM of target molecules.

14. The method of claim 1, further comprising:
comparing the levitation height of the density-amplified substrate to the levitation height of the diamagnetic substrate, target binding molecules-attached substrate, or target-bound substrate.

15. A kit comprising:
a paramagnetic liquid;
diamagnetic substrates;
one or more target binding molecules attached to or to be attached to the surface of said diamagnetic substrates, said target binding molecules selectively binding to target molecules to be detected; and
a density amplification material or precursor thereof for growing said density amplification material onto said diamagnetic object after binding the target molecules to be detected to said one or more target binding molecules attached to the diamagnetic substrates.

16. The kit of claim 15, wherein said paramagnetic liquid comprises a solution including paramagnetic salt in a solvent.

17. The kit of claim 15, wherein said target binding molecules are antigens and said target molecules are antibodies or antibody fragments.

18. The kit of claim 15, wherein said target binding molecules are antibodies or antibody fragments and said target molecules are antigens.

19. The kit of claim 15, wherein said target binding molecules are nucleic acids and said target molecules are complementary nucleic acids.

20. The kit of claim 15, wherein said target binding molecules are proteins and said target molecules are complementary proteins.

21. The kit of claim 17, wherein said antigens include HIV p24 antigen, Syphlis p41 antigen, Hepatitis Core antigen, haptens of antibiotics, haptens of penicillin, haptens of ampicillin, haptens of chloramphenicol or combinations thereof.

22. The kit of claim 17, wherein said antibody include HIV, Syphilis, Hepatitis C, rubella, penicillin, ampicillin, chloramphenicol, or combinations thereof.

23. The kit of claim 15, wherein said density amplification material is gold, silver, iron, mercury, nickel, copper, platinum, palladium, cobalt, iridium ions, polymer or mixtures thereof.

24. The kit of claim 15, wherein the kit is capable of detecting presence of target molecules in a sample at a concentration of less than 200 nM.

25. The kit of claim 15, wherein the kit is capable of detecting presence of target molecules in a sample at a concentration of less than 2 nM.

26. The kit of claim 15, wherein said density amplification material includes secondary moieties having a catalyst that promotes catalytic growth of said density amplification material.

27. The kit of claim 15, further comprising magnets.

28. The kit of claim 17, wherein said antigens are attached to the surface of said diamagnetic substrates.

29. The kit of claim 18, wherein said antibodies or antibody fragments are attached to the surface of said diamagnetic substrates.

30. The method of claim 3, wherein said antigens include HIV p24 antigen, Syphlis p41 antigen, Hepatitis Core antigen, haptens of antibiotics, haptens of penicillin, haptens of ampicillin, haptens of chloramphenicol or combinations thereof.

31. The method of claim 3, wherein said antibodies include HIV, Syphilis, Hepatitis C, rubella, penicillin, ampicillin, chloramphenicol, or combinations thereof.

32. The kit of claim 18, wherein said antigens include HIV p24 antigen, Syphlis p41 antigen, Hepatitis Core antigen, haptens of antibiotics, haptens of penicillin, haptens of ampicillin, haptens of chloramphenicol or combinations thereof.

33. The kit of claim 18, wherein said antibody include HIV, Syphilis, Hepatitis C, rubella, penicillin, ampicillin, chloramphenicol, or combinations thereof.
